(19) United States
(12) Patent Application Publication
(10) Pub. No.: US 2003/0212298 A1
(43) Pub. Date: Nov. 13, 2003

(54) Method of producing hexamethylene diamine from butadiene

(75) Inventors: Claudia Brasse, Hanau (DE); Thomas Haas, Frankfurt (DE); Robert Weber, Euskirchen (DE); Jurgen Neuroth, Frankfurt (DE)

Correspondence Address: FITCH, EVEN, TABIN & FLANNERY, SUITE 401L, 1801 K STREET, NW, WASHINGTON, DC, 20006-1201, US

(73) Assignee: Degussa AG

(21) Appl. No.: 10/396,688

(22) Filed: Mar. 26, 2003

(30) Foreign Application Priority Data
Mar. 28, 2002 (EP) 02 007 121.3

(51) Int. Cl. C 07C 2 9/48

(52) U.S. Cl. 564/492

(57) ABSTRACT

The invention relates to the production of hexamethylene diamine from butadiene. The method comprises the successively performed stages of: (i) a catalytic epoxidation of butadiene to 1,2-epoxy-3-butene; (ii) a basically catalyzed addition of hydrogen cyanide to butadiene monoxide to form a reaction mixture containing 3-hydroxy-4-pentene nitrile (3HPN) and 2-hydroxymethyl-3-butene nitrile (2HMBN); (iii) an acidically catalyzed dehydration of the cyanohydrines 3HPN and 2HMBN of stage (ii) to cis/trans-pentadiene nitrile (PDN); (iv) a basically catalyzed addition of the products of stage (iii) to form cis/trans-1,4-dicyanobutene-1 and -2 (DCB); and (v) a catalytic hydrogenation of the isomeric cis/trans-1,4-dicyanobutene of stage (iv) to hexamethylene diamine.

### CROSS REFERENCE TO RELATED APPLICATIONS

The present application claims priority to German Application No. 02 007 121.3, filed Mar. 28, 2002, which is incorporated in its entirety herein by reference.

### FIELD OF THE INVENTION

The present invention is directed to a multistage method of producing hexamethylene diamine (HMDA) from butadiene. In the first stage of the method, butadiene is converted into 1,2-epoxy-3-butene (butadiene monoxide). There are then two hydrogen cyanide addition stages, a dehydration stage and a hydrogenation stage.

### BACKGROUND

Hexamethylene diamine (HMDA) is a chemical intermediate produced in large amounts for industry and used in many different processes, including those concerned with the manufacture of nylon, plastics, urethane coatings, polyamides for printing inks, dimer acids, textiles, and biocides. A survey of methods for producing this intermeduate can be found in the Chemical Economics Handbook—SRI International, February 2000, No. 664.1000, G to J. In one method, HMDA is made from cyclohexane by carrying out an oxidation step to form adipic acid, esterifying the adipic acid, performing a hydrogenation step to form 1,6-hexane diol, reacting the 1,6-hexane diol with ammonia and forming HMDA. Alternatively, the adipic acid made from cyclohexane can be converted by amination and hydrogenation, first into adiponitrile and, then, by catalytic hydrogenation, to hexamethylene diamine. Adiponitrile can also be formed by the electrochemical dimerization of acrylonitrile and then undergo hydrogenation.

HMDA can be also be obtained using butadiene as the raw material. In this process, the butadiene is first converted by means of oxygen and hydrogen chloride into a dichlorobutene isomeric mixture. The mixture is then reacted with an alkali cyanide to form a dicyanobutene isomeric mixture, which is hydrogenated via the intermediate stage adiponitrile to form hexamethylene diamine. Unfortunately, a large amount of salt accumulates with this method and this makes it unattractive for large-scale use.

An alternative route for producing hexamethylene diamine, involves reacting butadiene, in the presence of a nickel catalyst having a phosphine-phosphite ligand, with hydrogen cyanide to form an isomeric mixture of 1-cyanobutene-2 and 3-cyanobutene -1. This mixture is converted by a 2-stage isomerization in the presence of various nickel catalysts into 1-cyanobutene-3 (1-pentene nitrile). Adiponitrile is formed by the addition of hydrogen cyanide to the 1-cyanobutene-3 in the presence of a homogeneous nickel catalyst, from which adiponitrile hexamethylene diamine is produced by catalytic hydrogenation. This method has the significant disadvantage that it uses homogeneous nickel catalysts which are difficult to regenerate. The method is also unattractive in that significant expense is required in order to avoid work-related hygienic problems.

U.S. Pat. No. 2,488,913 teaches a method of producing 1,4-dicyanobutene that can be hydrogenated to hexamethylene diamine. For example, 1,2-epoxybutene-3 can be reacted in the presence of a catalytic system of copper (I) chloride, copper, hydrogen chloride and ammonium chloride in aqueous phase with hydrogen cyanide to form the 1,4-dicyanobutene. However, based upon experiments conducted by the present inventors in which an attempt was made to repeat the method described in U.S. Pat. No. 2,488,913, it appears that the desired 1,4-dicyanobutene-2 is formed only to a slight extent.

U.S. Pat. No. 2,473,486 teaches a method of producing 1-cyano-1,3-butadiene (pentadiene nitrile) that comprises an addition of hydrogen cyanide to 1,2-epoxy-3-butene. According to the method described, hydrogen cyanide is formed by reacting sodium cyanide with carbon dioxide in the reaction mixture in situ. Aside from a small amount of pentadiene nitrile, the reaction mixture contains 1-cyano-3-butene-2-ol (=3-hydroxy-4-pentene nitrile). The reaction mixture is azylated by an anhydride and the reaction mixture obtained in this manner pyrolyzed at 350-700° C., during which pentadiene nitrile is formed.

### SUMMARY OF THE INVENTION

The present invention is based upon the development of a new method for producing hexamethylene diamine from butadiene that avoids many of the problems associated with methods that have been previously used. The method should not comprise more than 5 steps, and avoids the use of homogeneous nickel catalysts for the addition of hydrogen cyanide to an olefinic substrate.

Accordingly, the invention is directed to a method for the production of hexamethylene diamine that is characterized by the following successively performed stages: (i) catalytic epoxidation of butadiene to 1,2-epoxy-3-butene (butadiene monoxide); (ii) basically catalyzed addition of hydrogen cyanide to butadiene monoxide to form a reaction mixture containing 3-hydroxy-4-pentene nitrile (3HPN) and 2-hydroxymethyl-3-butene nitrile (2HMBN); (iii) acidically catalyzed dehydration of the cyanohydrines 3HPN and 2HMBN of stage (ii) to cis/trans-pentadiene nitrile (PDN); (iv) basically catalyzed addition of hydrogen cyanide to cis/trans-pentadiene nitrile of stage (iii) to form cis/trans-1,4-dicyanobutene-1 and -2 (DCB); and (v) catalytic hydrogenation of the isomeric cis/trans-1,4-dicyanobutene of stage (iv) to hexamethylene diamine.

In a preferred embodiment, the addition of hydrogen cyanide in stage (ii) is carried out in the presence of a catalyst selected from the group consisting of: alkali hydroxides; alkali cyanides; alkaline earth hydroxides, except for calcium hydroxide in aqueous phase; alkaline earth cyanides; basic ion exchangers in the OH form; and aliphatic and carbocyclic secondary or tertiary amines. The most preferred catalyst is one selected from the group consisting of: lithium hydroxide; lithium cyanide formed in situ; and an N-alkylated, cyclic amine with 5 or 6 ring members, wherein “alkyl” in the term “N-alkylated” stands for methyl, ethyl, n-propyl or n-butyl, and the cyclic amine with 5 or 6 members contains another heteroatom, i.e. other than the amine nitrogen. The addition of hydrogen cyanide may be carried out in the presence of an aprotic, dipolar solvent with the most preferred solvent being selected from: acetonitrile, N-methyl pyrrolidone or dimethyl formamide. In still other preferred embodiments, the butadiene monoxide in step (ii) is present in an excess over said hydrogen cyanide.

The dehydration of cyanohydrines in step (iii) is preferably carried out in the presence of an acidic fixed bed catalyst. It is also desirable that this dehydration be carried out in the presence of a polymerization inhibitor.

With respect to step (iv), the addition of hydrogen cyanide to cis/trans-pentadiene nitrile is preferably out in the presence of a catalyst selected from the group consisting of: a tertiary amine, an alkali hydroxide, an alkali cyanide, a secondary or tertiary alkali phosphate; and an alkali pyrophosphate. Especially preferred are alkali pyrophosphates (particularly potassium pyrophosphate) and secondary phosphates (particularly dipotassium hydrogen phosphate).

Preferred catalysts for the hydrogenation of the cis/trans-dicyanobutenes to hexamethylene diamine in step (v) are heterogeneous hydrogenation catalysts with one or more hydrogenation-active metals from the family of the 8th subgroup of the periodic table, especially from the Co, Ni, Ru, Pd and Pt family.

### DETAILED DESCRIPTION OF THE INVENTION

The present invention is directed to the synthetic method described above and includes stages (i)-(v), also referred to as steps a)-e). Reaction stage (i) involves the epoxidation of 1,3-butadiene. For example, butadiene can be expoxidized by a selective gaseous phase reaction using oxygen in the presence of a basic or silver catalyst (see e.g., U.S. Pat. No. 6,172,245; U.S. Pat. No. 6,018,061 and U.S. Pat. No. 5,756,779). The butadiene used can be purchased commercially or made using procedures well known in the art.

The attachment of hydrogen cyanide to butadiene monoxide in the presence of a basic catalyst is preferably carried out in the presence of a catalyst from the series of: alkali hydroxides; alkali cyanides; alkaline earth hydroxides, except for calcium hydroxide in aqueous phase; alkaline earth cyanides; basic ion exchangers in the OH form; and carbocyclic secondary or tertiary amines. In an especially preferred embodiment of stage (ii), the catalyst used is lithium hydroxide, lithium cyanide formed in situ or an N-alkylated, cyclic amine with 5 or 6 ring members in which alkyl stands for methyl, ethyl, n-propyl or n-butyl and the ring can additionally contain another heteroatom.

The base-catalyzed addition of HCN in stage (ii) of the invention takes place chemoselectively at the epoxide function and with high regioselectivity to the C-1 atom. The formation of 3-hydroxy-4-pentene nitrile (3-HPN) thus predominates over the formation of 2-hydroxymethyl-3-butene nitrile (2-HMBN).

The addition of HCN to butadiene monoxide preferably takes place in an organic solvent, that can also, however, contain water. It was found that the addition of HCN with high regioselectivity regarding the ratio of 3-HPN to 2-HMBN can be obtained if the reaction is carried out in the presence of an aprotic solvent such as acetonitrile, dimethyl formamide, or N-methyl-pyrrolidone. The addition of HCN advantageously takes place at elevated temperature, especially at a temperature in the range of 50-150° C.

In an especially preferred embodiment, the addition of HCN to butadiene monoxide takes place using an aqueous solution of lithium hydroxide as catalyst, at a temperature in the range of 70-120° C. and in the presence of dimethyl formamide as solvent. Instead of the aqueous lithium hydroxide, the catalyst used can also be a lithium cyanide formed in situ in dissolved or suspended form.

The reaction mixture containing the cyanohydrines 3-HPN and 2-HMBN, or the cyanohydrines 3-HPN and 2-HMBN isolated from this mixture, are used for the dehydration of stage (iii). To this end, the basic catalyst, is neutralized or removed and replaced by an acidic dehydration catalyst. The dehydration of the cyanohydrines takes place in an organic solvent, and water formed during this procedure can be drawn off as an azeotrope. Suitable catalysts are organic acids, e.g., sulfonic acids such as methane sulfonic acid and toluene sulfonic acid, or ion exchangers with sulfonic acid groups or phosphonic acid groups. In an alternative embodiment, inorganic solids such as acidically acting oxides and silicates, especially zeolites such as zeolite ZSM5, as well as oxides, e.g., aluminum oxide, coated with an inorganic acid such as phosphoric acid or its anhydrides, are used as acidic catalysts. For example, Al₂O₃ treated with phosphoric acid, that was subsequently calcined, may be used.

The dehydration of cyanohydrines as described herein can be carried out at a significantly lower temperature than required in the pyrolysis of acylated cyanohydrines according to U.S. Pat. No. 2,473,486. The dehydration advantageously takes place at a temperature of below 150° C. on a solid dehydration catalyst. A fixed bed dehydration catalyst is preferably operated in a trickle bed manner, during which the cyanohydrines dissolved in an organic solvent are advantageously conducted over the catalyst. Preferably, the solution containing the cyanohydrines additionally contains a polymerization inhibitor such as hydroquinone, hydroquinone monomethyl ether or a tertiary butylated phenol in an effective amount.

Hydrogen cyanide is attached to the pentadiene nitrile in stage (iv) of the procedure. Suitable catalysts include: tertiary amines, alkali hydroxides, alkali cyanides, secondary and tertiary alkali phosphates and alkali pyrophosphates. The attachment of HCN to pentadiene nitrile preferably takes place in the presence of a weakly basic catalyst. This is because, in the presence of strongly basic catalysts, aside from the desired cis/trans-1,4-dicyanobutene-2 and its isomer cis/trans-1,4-dicyanobutene-1,1,2,4-tricyanobutane, unwanted isomers can be formed by further HCN attachment. In addition, isomers of decatriene dinitrile and other HCN adducts can result from dimerization.

According to an especially preferred embodiment, the addition of HCN to cis/trans-pentadiene nitrile in stage (iv) is catalyzed using: alkali pyrophosphate (especially potassium pyrophosphate) or a secondary phosphate (especially dipotassium hydrogen phosphate). Using these catalysts, especially potassium pyrophosphate, results in isomeric cis/trans/1,4-dicyanobutenes with a selectivity approaching 100%. The designation cis/trans-pentadiene nitrile as well as cis/trans-1,4-dicyanobutene-1 and -2 signifies that compounds can be in an isomeric mixture or particular cis or trans isomers.

The cis/trans-1,4-dicyanobutene-1 and -2 formed in stage (iv) can be hydrogenated using known hydrogenating catalysts for nitrile groups and olefinic double bonds to form hexamethylene diamine (stage (v)). This preferably takes place in the presence of a heterogeneous catalyst with one or more hydrogenation-active metals from the family of the 8th subgroup of the periodic table. Especially suitable catalysts contain as the hydrogenation-active component one or more metals from the series cobalt, nickel, ruthenium, palladium and platinum. The catalyst can be used in the form of a suspension or as a fixed bed catalyst. If a fixed bed reactor is used, it may be operated using either a bubble method or a trickle bed method. The hydrogenation takes place at a partial hydrogen pressure in the range of 2 to 20 MPa, preferably 3 to 10 MPa. It can be carried out at a temperature in the range of 30 to approximately 150° C., preferably 70 to 120° C. The hydrogenation is preferably carried out in the presence of an organic solvent that may, also preferably, contain a polymerization inhibitor.

### EXAMPLES

### Reference Example 1 (VB1)

### Production of 1,4-dicyanobutene according to U.S. Pat. No. 2,488,913

Butadiene monoxide and hydrogen cyanide were used in a molar ratio of 1 to 2. The catalytic system consisted of CuCl, Cu, HCl (36%), HN4Cl and water in the amounts specified by the patent. After 2h at 75-80° C. the reaction mixture was extracted with toluene. According to gas-chromatographic analysis, the reaction mixture freed of solvent contained only 8% by area 1,4-dicyanobutene, especially 1,4-dicyanobutene-2.

### Reference Example 2 (VB2)

### Production According to U.S. Pat. No. 4,448,913 (example 3)

1,2-epoxybutene-3 (commercially available) was reacted with hydrogen cyanide in the presence of calcium hydroxide in aqueous phase under the conditions indicated in the US patent. After 2 h no reaction with HCN was observed.

### Examples 1 to 11

### Production of 3-HPN and 2HMBN from Butadiene Monoxide and HCN

1,2-epoxybutene-3 was reacted in acetonitrile (examples 1 to 3) or dimethyl formamide (examples 4 to 11) with hydrogen cyanide 3 h at 70° C. (examples 1 to 7) and 3 h at 120° C. (examples 8 to 11).

The molar ratio of butadiene monoxide (bumo) to HCN was always 1 to 1.1. The catalysts used are shown in the following tables. The amount of catalyst used was 0.5 to 0.9 molar % in examples 1 to 3 and 0.8 to 1.2 molar % in examples 4 to 11.

TABLE 1
Solvent acetonitrile, T = 70° C.

| Example | | Conversion | Selectivity | Regioselectivity |
|---|---|---|---|---|
| No. | Catalyst | (%) | 3HPN (%) | (%) 3HPN:2HMBN |
| 1 | LiOH | 7.7 | 35.5 | 91.8 |
| 2 | KOH | 11.3 | 37.8 | 91.8 |
| 3 | JRA400 | 6.3 | 16.4 | 95.7 |
| | (OH) | | | |

T = 70° C.; t = 3 h; 5 ml acetonitrile; 7.5 mMol bumo

TABlE 2
Solvent DMF,

| Ex. | | Conversion | Selectivity | Regioselectivity |
|---|---|---|---|---|
| No. | Catalyst | (%) | 3HPN (%) | (%) 3HPN:2HMBN |
| 4 | LiOH | 45.7 | 34.7 | 99.6 |
| 5 | LiOH 12.7% in | 90.1 | 74.9 | 99.5 |
| | water | | | |
| 6 | KOH | 43.1 | 28.7 | 99.5 |
| 7 | N-methyl | 54.8 | 42.7 | 99.9 |
| | pyrrolidine | | | |
| 8 | LiOH | 93.9 | 78.5 | 99.4 |
| 9 | KOH | 88.9 | 74.1 | 99.1 |
| 10 | JRA400[OH] | 87.3 | 71.7 | 99.2 |
| 11 | Ca(OH)₂ | 81.6 | 69.9 | 98.7 |

T = 70° C. (ex. 4-ex.7), and T = 120° C. (ex. 8-ex. 11)

### Example 12

### Dehydration of 3HPN to Pentadiene Nitrile

3HPN was boiled in a toluene solution in the presence of p-toluenesulfonic acid under reflux. Water formed was separated azeotropically. Pentadiene nitrile forms as a mixture of the cis and trans isomers.

### Example 13

### Production of 1,4-dicyanobutenes (DCB) by the Addition of HCN to Pentadiene Nitrile (PDN)

Pentadiene nitrile and HCN were reacted in acetonitrile at a molar ratio of HCN:PDN:catalyst of 1:1.015:(0.1-0.1). Results are shown in Table 3.

TABLE 3

| | | Conversion | Selectivity | | |
|---|---|---|---|---|---|
| Ex. No. | Catalyst | PDN (%) | DCB (%) | T (° C.) | t (f) |
| 13*) | LiOH | 58.7 | 39.9 | 40 | 64 |
| 14 | triethylamine | 16.6 | 65.6 | 40 | 64 |
| 15 | N-methyl | 20 | 59.6 | 40 | 64 |
| | pyrrolidine | | | | |
| 16 | potassium | 24.5 | 98.8**) | 80 | 2, |
| | pyrophosphate | | | 25 | then 22, |
| | | | | 80 | then 2 |
| 17 | N-methyl | 10.0 | 53.2 | 80 | 2, |
| | pyrrolidine | | | 25 | then 22, |
| | | | | 80 | then 2 |

*)Yield of DCB 23.4%, additionally produced 22.5% byproducts, especially tricyanobutane + tricyanooctadiene isomers
**)Approximately 80% cis/trans DCB-2 and 20% cis/trans DCB-1 were formed.

All references cited herein are fully incorporated by reference. Having now fully described the invention, it will be understood by one of ordinary skill in the art that the invention may be performed within a wide and equivalent range of conditions, parameters and the like, without affecting the spirit or scope of the invention or any embodiment thereof.

What is claimed is:

1. A method of producing hexamethylene diamine from butadiene, comprising:
a) forming 1,2-epoxy-3-butene (butadiene monoepoxide) by the catalytic epoxidation of butadiene;
b) forming a reaction mixture containing 3-hydroxy-4-pentene nitrile (3HPN) and 2-hydroxymethyl-3-butene nitrite (2HMBN) by the basically catalyzed addition of hydrogen cyanide to the butadiene monoepoxide of step a);
c) forming cis/trans-pentadiene nitrile (PDN) by the acidically catalyzed dehydration of the cyanohydrines 3HPN and 2HMBN of step b);
d) forming cis/trans-1,4-dicyanobutene-1 and/trans-1,4-dicyanobutene-2 by the basically catalyzed addition of hydrogen cyanide to the cis/trans-pentadiene nitrile of step c); and
e) forming said hexamethylene diamine by the catalytic hydrogenation of the isomeric cis/trans-1,4-dicyanobutene of step d).

2. The method of claim 1, wherein the addition of hydrogen cyanide in step b) is carried out in the presence of a catalyst selected from the group consisting of: alkali hydroxides; alkali cyanides; alkaline earth hydroxides except for calcium hydroxide in aqueous phase; alkaline earth cyanides; basic ion exchangers in the OH form; and aliphatic and carbocyclic secondary or tertiary amines.

3. The method of claim 2, wherein the catalyst used is selected from the group consisting of: lithium hydroxide; lithium cyanide formed in situ; and an N-alkylated, cyclic amine with 5 or 6 ring members, wherein the alkyl in the term “N-alkylated” stands for methyl, ethyl, n-propyl or n-butyl, and said cyclic amine with 5 or 6 members contains another heteroatom.

4. The method of claim 3, wherein the addition of hydrogen cyanide in step b) is carried out in the presence of an aprotic, dipolar solvent.

5. The method of claim 4, wherein said aprotic dipolar solvent is selected from the group consisting of: acetonitrile, N-methyl pyrrolidone or dimethyl formamide.

6. The method of claim 5, wherein said butadiene monoepoxide in step b) is present in an excess over said hydrogen cyanide.

7. The method of any one of claims 1-6, wherein the dehydration of the cyanohydrines in step c) is carried out in the presence of an acidic fixed bed catalyst.

8. The method of claim 7, wherein the dehydration of step c) is carried out in the presence of a polymerization inhibitor.

9. The method of claim any one of claims 1-6, wherein the addition of hydrogen cyanide to cis/trans-pentadiene nitrile in step d) is carried out in the presence of a catalyst selected from the group consisting of: a tertiary amine, an alkali hydroxide, an alkali cyanide, a secondary or tertiary alkali phosphate; and an alkali pyrophosphate.

10. The method of claim 9, wherein said catalyst is either an alkali pyrophosphate, or a secondary phosphate.

11. The method of claim 10, wherein said catalyst is either potassium pyrophosphate or dipotassium hydrogen phosphate.

12. The method of any one of claims 1-6, wherein the hydrogenation of the cis/trans-dicyanobutenes to hexamethylene diamine in step e) takes place in the presence of a heterogeneous hydrogenation catalyst with one or more hydrogenation-active metals from the family of the 8th subgroup of the periodic table.

13. The method of claim 12, wherein said hydrogen-active metal is selected from the Co, Ni, Ru, Pd or Pt family.
